(19) United States
(12) Patent Application Publication
(10) Pub. No.: US 2015/0238156 A1
(43) Pub. Date: Aug. 27, 2015

(54) RADIATION GENERATING APPARATUS

(71) Applicant: Metal Industries Research & Development Centre, Kaohsiung (TW)

(72) Inventors: Yen-Chun Chen, Kaohsiung City (TW); Wei-Hung Shih, Kaohsiung City (TW); Yi-San Chang, Tainan City (TW); Tsung-Chih Yu, Tainan City (TW); Ming-Hui Cheng, Kaohsiung City (TW)

(73) Assignee: Metal Industries Research & Development Centre, Kaohsiung (TW)

(21) Appl. No.: 14/190,101

(22) Filed: Feb. 26, 2014

(51) Int. Cl. A61B 6/00

(52) U.S. Cl. CPC A61B 6/40

(57) ABSTRACT

A radiation generating apparatus includes a target base, a target, a holding assembly and an electronic beam generating device. The target is disposed on the target base. The holding assembly holds the target base. The electronic beam generating device is adapted to generate an electronic beam, wherein the electronic beam is emitted to the target to generate a radiation. The target, the holding assembly and the electronic beam generating device are located at the same side of the target base.

### BACKGROUND

1. Technical Field

The invention relates to a radiation generating apparatus. Particularly, the invention relates to a radiation generating apparatus capable of using an electronic beam to irradiate a target to generate radiation.

2. Related Art

An X-ray tube is an image device capable generating X-ray, which can be applied in fields of industrial testing, medical diagnosis or medical treatment. Generally, the X-ray tube includes an electronic beam generating device and a target, where the electronic beam generating device can be composed of a high-voltage power supplier and a tungsten filament. When the high-voltage power supplier supplies enough current to the tungsten filament, the tungsten filament generates an electronic beam, and the electronic beam is emitted to the target to generate the X-ray.

In the aforementioned operation process, most of the energy of the electronic beam emitted to the target is converted into heat to increase the temperature of the target. In this way, under a high-power operation, the high-energy electronic beams that continuously strike the X-ray target may cause overheat and wear of the X-ray target to decrease a service life of the X-ray target. Moreover, in some designs of the X-ray tube, besides that the electronic beam generating device and the target are included, components such as a cooling system used for cooling the target are also included, such that the X-ray tube has a larger volume and is not complied with user's requirement.

### SUMMARY

The invention is directed to a radiation generating apparatus, which has a smaller volume and overheat of a target thereof is avoided.

The invention provides a radiation generating apparatus including a target base, a target, a holding assembly and an electronic beam generating device. The target is disposed on the target base. The holding assembly holds the target base. The electronic beam generating device is adapted to generate an electronic beam, where the electronic beam is emitted to the target to generate a radiation. The target, the holding assembly and the electronic beam generating device are located at a same side of the target base.

In an embodiment of the invention, the radiation generating apparatus further includes a first driving unit, where the holding assembly has an axial direction and a radial direction, the electronic beam is emitted to the target along the axial direction to generate the radiation, and the first driving unit is adapted to drive the target base to move along the radial direction.

The invention provides a radiation generating apparatus including a target base, a target, a holding assembly, an electronic beam generating device and a first driving unit. The target is disposed on the target base. The holding assembly holds the target base and has an axial direction and a radial direction. The electronic beam generating device is adapted to generate an electronic beam, where the electronic beam is emitted to the target along the axial direction to generate a radiation. The first driving unit is adapted to drive the target base to move along the radial direction.

In an embodiment of the invention, the target, the holding assembly and the electronic beam generating device are located at a same side of the target base.

In an embodiment of the invention, the first driving unit is disposed on the holding assembly, and is adapted to drive the holding assembly to move along the radial direction.

In an embodiment of the invention, the holding assembly includes a second driving unit and a rotation member. The rotation member is connected between the second driving unit and the target base, and the second driving unit is adapted to drive the rotation member and the target base to rotate along the axial direction.

In an embodiment of the invention, the rotation member includes a rotation shaft and a hollow housing. The rotation shaft is connected between the hollow housing and the second driving unit, and the hollow housing is connected to the target base, and the target and the electronic beam generating device are located in the hollow housing.

In an embodiment of the invention, the radiation generating apparatus further includes a power supply unit and a connection element, where the power supply unit is disposed outside the hollow housing, the rotation shaft is a hollow shaft, the first driving unit is disposed in the hollow shaft, and the connection element penetrates through the hollow shaft to be connected between the electronic beam generating device and the power supply unit.

In an embodiment of the invention, the rotation member is a rotation shaft, the target is ring-shaped and surrounds the rotation shaft, and the second driving unit is adapted to drive the rotation shaft and the target base to rotate along the axial direction.

In an embodiment of the invention, the first driving unit is adapted to drive the target base to vibrate along the radial direction.

In an embodiment of the invention, the target is an X-ray target, the radiation is an X-ray.

In an embodiment of the invention, the radiation penetrates through the target base to be emitted out.

According to the above descriptions, in the radiation generating apparatus of the invention, the target, the holding assembly and the electronic beam generating device are all disposed at the same side of the target base other than respectively disposed at two opposite sides of the target base, by which a volume of the radiation generating apparatus is effectively decreased, so that the radiation generating apparatus occupies less space to cope with user's requirement. Moreover, when the electronic beam generated by the electronic beam generating apparatus is emitted to the target along the axial direction of the holding assembly, besides that the target can be driven by the second driving unit to rotate along the axial direction, and the target can also be driven by the first driving unit to continuously move along the radial direction, so as to continuously change a region of the target struck by the electronic beam. In this way, a time period that each region of the target is not struck by the electronic beam is increased to improve a cooling efficiency thereof, so as to avoid overheat of the target due to strike of the electronic beam, and prolong a service life of the target.

In order to make the aforementioned and other features and advantages of the invention comprehensible, several exemplary embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic diagram of a radiation generating apparatus according to an embodiment of the invention.

FIG. 2 is a schematic diagram illustrating a trajectory that an electronic beam strikes a target of FIG. 1.

FIG. 3 is a schematic diagram illustrating a trajectory that an electronic beam strikes a target according to another embodiment of the invention.

FIG. 4 is a schematic diagram of a radiation generating apparatus according to another embodiment of the invention.

### DETAILED DESCRIPTION OF DISCLOSED EMBODIMENTS

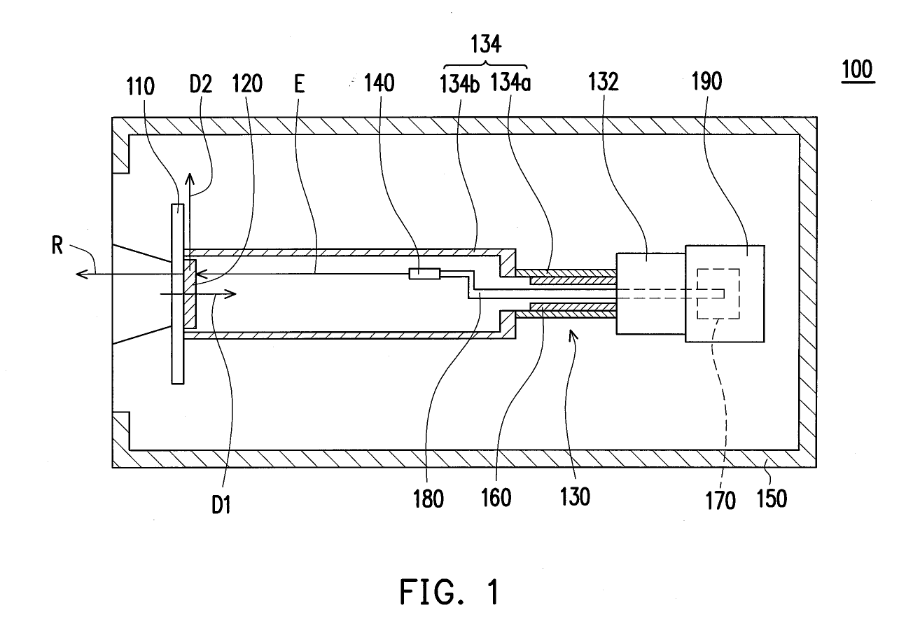

FIG. 1 is a schematic diagram of a radiation generating apparatus according to an embodiment of the invention. Referring to FIG. 1, the radiation generating apparatus 100 of the present embodiment is, for example, a transmission type X-ray tube applied for industrial testing, medical diagnosis or medical treatment, and includes a target base 110, a target 120, a holding assembly 130, an electronic beam generating device 140 and a tube 150. The tube 150 is, for example, a vacuum tube suitable for the X-ray tube, and the holding assembly 130 is disposed in the tube 150 and holds the target base 110. The target 120 is, for example, an X-ray target and is disposed on the target base 110. The electronic beam generating device 140 is disposed in the tube 150 and is adapted to generate an electronic beam E, where the electronic beam E is emitted to the target 120 along an axial direction D1 of the holding assembly 130 to generate a radiation R such as an X-ray, and the radiation R penetrates through the target base 110 to be emitted out.

As shown in FIG. 1, the target 120, the holding assembly 130 and the electronic beam generating device 140 are all located at the same side (illustrating as the right side of the target base 110) of the target base 110 other than respectively disposed at two opposite sides of the target base 110, by which a volume of the radiation generating apparatus 100 is effectively decreased, so that the radiation generating apparatus 100 occupies less space to cope with user's requirement.

In the present embodiment, the radiation generating apparatus 100 further includes a first driving unit 160, where the first driving unit 160 is disposed on the holding assembly 130, and is adapted to drive the holding assembly 130 and the target base 110 to move along a radial direction D2 of the holding assembly 130. Moreover, the holding assembly 130 includes a second driving unit 132 and a rotation member 134, where the rotation member 134 is connected between the second driving unit 132 and the target base 110, and the second driving unit 132 is adapted to drive the rotation member 134 and the target base 110 to rotate along the axial direction D1 of the holding assembly 130.

Under the aforementioned actuation method, when the electronic beam E generated by the electronic beam generating device 140 is emitted to the target 120 along the axial direction D1, besides that the target 120 can be driven by the second driving unit 132 to rotate along the axial direction D1, and the target 120 can also be driven by the first driving unit 160 to continuously move along the radial direction D2, so as to continuously change a region of the target 120 struck by the electronic beam E. In this way, a time period that each region of the target 120 is not struck by the electronic beam E is increased to improve a cooling efficiency, so as to avoid overheat of the target 120 due to strike of the electronic beam E, and prolong a service life of the target 120.

In detail, the rotation member 134 of the present embodiment includes a rotation shaft 134a and a hollow housing 134b. The rotation shaft 134a is connected between the hollow housing 134b and the second driving unit 132, the hollow housing 134b is connected to the target base 110, and the target 120 and the electronic beam generating device 140 are located in the hollow housing 134b. The hollow housing 134b is, for example, an insulation housing to prevent current leakage of the electronic beam generating device 140.

The radiation generating apparatus 100 further includes a power supply unit 170 and a connection element 180, where the power supply unit 170 is disposed outside the hollow housing 134b, the rotation shaft 134a is a hollow shaft, the first driving unit 160 is disposed in the hollow shaft to drive the rotation member 134 and the target base 110 to rotate, and the connection element 180 penetrates through the hollow shaft to be connected between the electronic beam generating device 140 and the power supply unit 170. The connection element 180 is used to hold the electronic beam generating device 140 and includes a circuit, and the electronic beam generating device 140 is electrically connected to the power supply unit 170 through the circuit. The power supply unit 170 is, for example, disposed in a holding structure 190, and the holding structure 190 is fixed to the tube 150 of the radiation generating apparatus 100 and is connected to the holding assembly 130, so as to hold the holding assembly 130 and the target base 110. The second driving unit 132 is adapted to drive the rotation shaft 134a to rotate, so as to drive the hollow housing 134b, the target base 110 and the first driving unit 160 to rotate along the axial direction D1, and the electronic beam generating device 140, the connection element 180, the power supply unit 170 and the holding structure 190 are not rotated.

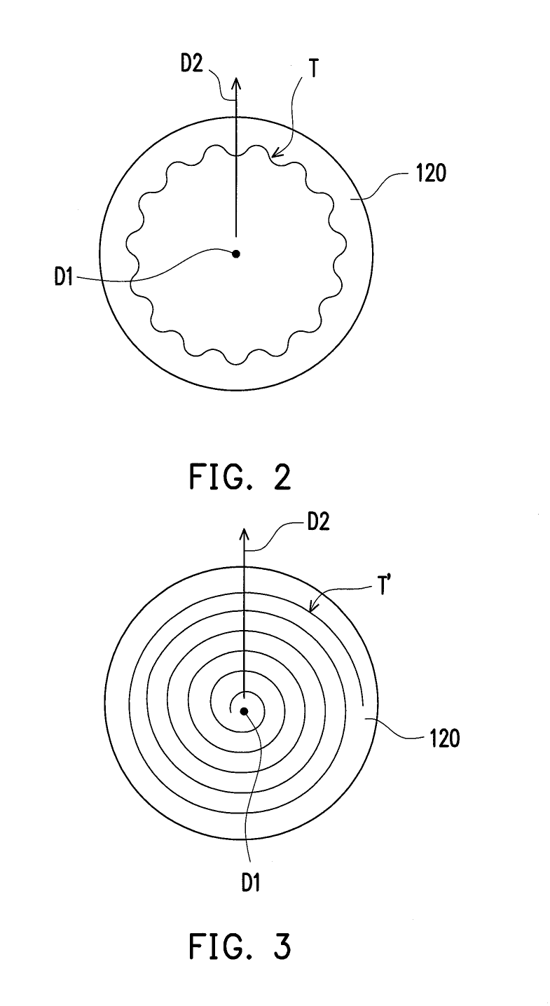

FIG. 2 is a schematic diagram illustrating a trajectory that the electronic beam strikes the target of FIG. 1. In the present embodiment, the first driving unit 160 is, for example, an oscillator and is adapted to drive the target base 110 to vibrate along the radial direction D2, and in collaboration with rotation of the target base 110 along the axial direction D1, the trajectory T that the electronic beam E strikes the target 120 is shown in FIG. 2, where the trajectory T is a continuous reciprocating trajectory along the radial direction D2. Further, a damping can be configured between the first driving unit 160 and an inner wall of the rotation shaft 134a, which is not limited by the invention. Moreover, the method that the first driving unit 160 drives the target base 110 and the target 120 is not limited by the invention, which is described below with reference of figures.

FIG. 3 is a schematic diagram illustrating a trajectory that the electronic beam strikes the target according to another embodiment of the invention. In the present embodiment, the first driving unit 160 is not an oscillator, and is adapted to drive the target base 110 to move along the radial direction D2 in an appropriate manner, and in collaboration with rotation of the target base 110 along the axial direction D1, the trajectory T that the electronic beam E strikes the target 120 is shown in FIG. 3. A moving range of the trajectory T′ along the radial direction D2 is larger, so as to improve a utilization rate of the target 120. In other embodiments, a rotation speed of the target base 110 rotated along the axial direction D1 and a moving manner of the target base 110 moving along the radial direction D2 can be changed according to an actual requirement, so as to adjust the trajectory that the electronic beam E strikes the target 120, which is not limited by the invention.

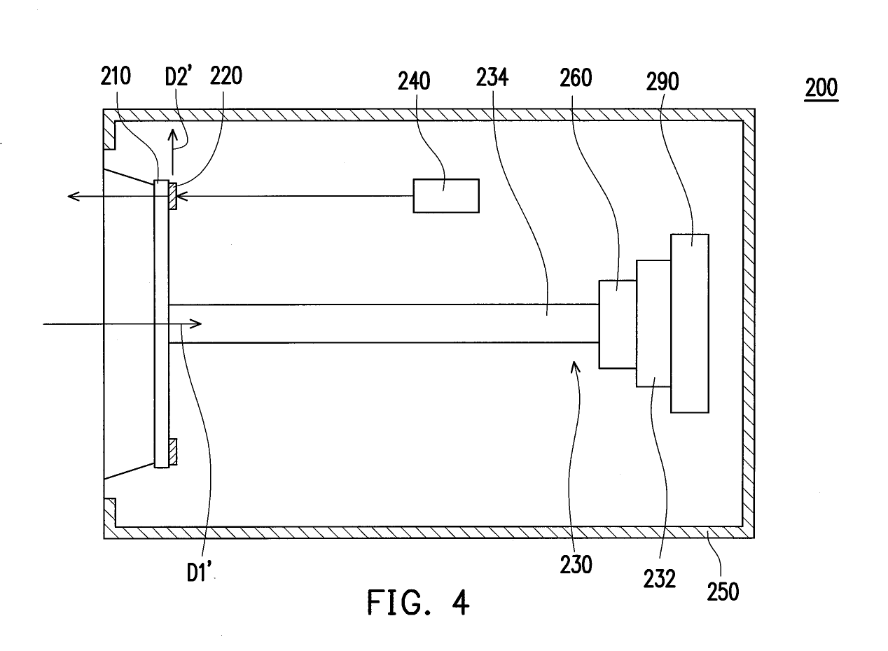

FIG. 4 is a schematic diagram of a radiation generating apparatus according to another embodiment of the invention. In the radiation generating apparatus 200 of FIG. 4, operations of a target base 210, a target 220, a holding assembly 230, an electronic beam generating device 240, a tube 250, a first driving unit 260 and a holding structure 290 are similar to operations of the target base 110, the target 120, the holding assembly 130, the electronic beam generating device 140, the tube 150, the first driving unit 160 and the holding structure 190, and details thereof are not repeated. Differences between the radiation generating apparatus 200 and the radiation generating apparatus 100 are that a rotation member 234 of the holding assembly 230 is a rotation shaft, the target 220 is ring-shaped and surrounds the rotation shaft, the electronic beam generating device 240 is disposed outside the holding assembly 230, and the first driving unit 260 is disposed outside the rotation shaft. When the first driving unit 260 drives the target base 210 to move along a radial direction D2′, the second driving unit 232 of the holding assembly 230 is adapted to drive the rotation shaft and the target base 210 to rotate along an axial direction D1′.

In summary, in the radiation generating apparatus of the invention, the target, the holding assembly and the electronic beam generating device are all disposed at the same side of the target base other than respectively disposed at two opposite sides of the target base, by which a volume of the radiation generating apparatus is effectively decreased, so that the radiation generating apparatus occupies less space to cope with user's requirement. Moreover, when the electronic beam generated by the electronic beam generating apparatus is emitted to the target along the axial direction of the holding assembly, besides that the target can be driven by the second driving unit to rotate along the axial direction, and the target can also be driven by the first driving unit to continuously move along the radial direction, so as to continuously change a region of the target struck by the electronic beam. In this way, a time period that each region of the target is not struck by the electronic beam is increased to improve a cooling efficiency thereof, so as to avoid overheat of the target due to strike of the electronic beam, and prolong a service life of the target.

It will be apparent to those skilled in the art that various modifications and variations can be made to the structure of the invention without departing from the scope or spirit of the invention. In view of the foregoing, it is intended that the invention cover modifications and variations of this invention provided they fall within the scope of the following claims and their equivalents.

What is claimed is:

1. A radiation generating apparatus, comprising:
a target base,
a target, disposed on the target base;
a holding assembly, holding the target base; and
an electronic beam generating device, adapted to generate an electronic beam, wherein the electronic beam is emitted to the target to generate a radiation, and the target, the holding assembly and the electronic beam generating device are located at a same side of the target base.

2. The radiation generating apparatus as claimed in claim 1, further comprising a first driving unit, wherein the holding assembly has an axial direction and a radial direction, the electronic beam is emitted to the target along the axial direction to generate the radiation, and the first driving unit is adapted to drive the target base to move along the radial direction.

3. The radiation generating apparatus as claimed in claim 2, wherein the first driving unit is disposed on the holding assembly, and is adapted to drive the holding assembly to move along the radial direction.

4. The radiation generating apparatus as claimed in claim 2, wherein the holding assembly comprises a second driving unit and a rotation member, the rotation member is connected between the second driving unit and the target base, and the second driving unit is adapted to drive the rotation member and the target base to rotate along the axial direction.

5. The radiation generating apparatus as claimed in claim 4, wherein the rotation member comprises a rotation shaft and a hollow housing, the rotation shaft is connected between the hollow housing and the second driving unit, the hollow housing is connected to the target base, and the target and the electronic beam generating device are located in the hollow housing.

6. The radiation generating apparatus as claimed in claim 5, further comprising a power supply unit and a connection element, wherein the power supply unit is disposed outside the hollow housing, the rotation shaft is a hollow shaft, the first driving unit is disposed in the hollow shaft, and the connection element penetrates through the hollow shaft to be connected between the electronic beam generating device and the power supply unit.

7. The radiation generating apparatus as claimed in claim 4, wherein the rotation member is a rotation shaft, the target is ring-shaped and surrounds the rotation shaft, and the second driving unit is adapted to drive the rotation shaft and the target base to rotate along the axial direction.

8. The radiation generating apparatus as claimed in claim 2, wherein the first driving unit is adapted to drive the target base to vibrate along the radial direction.

9. The radiation generating apparatus as claimed in claim 1, wherein the target is an X-ray target, and the radiation is an X-ray.

10. The radiation generating apparatus as claimed in claim 1, wherein the radiation penetrates through the target base to be emitted out.

11. A radiation generating apparatus, comprising:
a target base;
a target, disposed on the target base;
a holding assembly, holding the target base and having an axial direction and a radial direction;
an electronic beam generating device, adapted to generate an electronic beam, wherein the electronic beam is emitted to the target along the axial direction to generate a radiation; and
a first driving unit, adapted to drive the target base to move along the radial direction.

12. The radiation generating apparatus as claimed in claim 11, wherein the target, the holding assembly and the electronic beam generating device are located at a same side of the target base.

13. The radiation generating apparatus as claimed in claim 11, wherein the first driving unit is disposed on the holding assembly, and is adapted to drive the holding assembly to move along the radial direction.

14. The radiation generating apparatus as claimed in claim 11, wherein the holding assembly comprises a second driving unit and a rotation member, the rotation member is connected between the second driving unit and the target base, and the second driving unit is adapted to drive the rotation member and the target base to rotate along the axial direction.

15. The radiation generating apparatus as claimed in claim 14, wherein the rotation member is a hollow housing, and the target and the electronic beam generating device are located in the hollow housing.

16. The radiation generating apparatus as claimed in claim 15, further comprising a power supply unit and a connection element, wherein the power supply unit is disposed outside the hollow housing, the rotation shaft is a hollow shaft, the first driving unit is disposed in the hollow shaft, and the connection element penetrates through the hollow shaft to be connected between the electronic beam generating device and the power supply unit.

17. The radiation generating apparatus as claimed in claim 14, wherein the rotation member is a rotation shaft, the target is ring-shaped and surrounds the rotation shaft, and the second driving unit is adapted to drive the rotation shaft and the target base to rotate along the axial direction.

18. The radiation generating apparatus as claimed in claim 12, wherein the first driving unit is adapted to drive the target base to vibrate along the radial direction.

19. The radiation generating apparatus as claimed in claim 1, wherein the target is an X-ray target, and the radiation is an X-ray.

20. The radiation generating apparatus as claimed in claim 11, wherein the radiation penetrates through the target base to be emitted out.
